(19) United States
(12) Patent Application Publication
(10) Pub. No.: US 2012/0241356 A1
(43) Pub. Date: Sep. 27, 2012

(54) HEAT- AND VIBRATION-STABLE INSULIN PREPARATIONS

(75) Inventors: Anja Pfenninger, Frankfurt am Main (DE); Norbert Tennagels, Frankfurt am Main (DE); Christiane Fuerst, Frankfurt am Main (DE)

(73) Assignee: SANOFI-AVENTIS DEUTSCHLAND GMBH, Frankfurt (DE)

(21) Appl. No.: 13/382,420

(22) PCT Filed: Jul. 2, 2010
(86) PCT No.: PCT/EP10/59430

§ 371 Date: May 4, 2012

Related U.S. Application Data
(60) Provisional application No. 61/264,358, filed on Nov. 25, 2009.

(30) Foreign Application Priority Data
Jul. 6, 2009 (DE) 10 2009 031 754.6

(51) Int. Cl. A61K 38/28; A61J 1/05; A61P 3/10

(52) U.S. Cl. 206/570; 514/5.9; 514/6.5; 514/6.4

(57) ABSTRACT

The invention relates to a method for producing an aqueous, pharmaceutical formulation comprising an insulin, an insulin analog, or an insulin derivative, wherein the ready-made formulation takes place directly by dissolving the insulin, the insulin analog, or the insulin derivative as a solid in a suitable solvent mixture.

The invention relates to a method for preparing an aqueous pharmaceutical formulation comprising an insulin, insulin analog or insulin derivative, the ready-to-use formulation being effected directly by dissolving the insulin, insulin analog or insulin derivative in solid form with a suitable solvent mixture.

An increasing number of people around the world suffer from diabetes mellitus. Many of them are what are called type I diabetics, for whom replacement of the deficient endocrine insulin secretion is the only possible therapy at present. Those affected are dependent on insulin injections for life, usually several times a day. Type II diabetes contrasts with type I diabetes in that there is not always a deficiency of insulin, but in a large number of cases, especially at the advanced stage, treatment with insulin, where appropriate in combination with an oral antidiabetic, is considered the most advantageous form of therapy.

In healthy individuals, release of insulin by the pancreas is strictly coupled to the blood glucose concentration. Elevated blood glucose levels, like those occurring after meals, are quickly compensated by a corresponding rise in insulin secretion. In the fasting state, the plasma insulin level falls to a base line value which is sufficient to ensure a continuous supply of glucose to insulin-sensitive organs and tissues, and to keep hepatic glucose production low in the night. The replacement of the endogenous insulin secretion by exogenous, usually subcutaneous administration of insulin does not in general come close to the above-described quality of the physiological regulation of blood glucose. Frequently there are instances of blood glucose being thrown off-track, either upwardly or downwardly, and in their most severe forms these instances may be life-threatening. In addition, however, blood glucose levels which are elevated over years, without initial symptoms, constitute a considerable health risk. The large-scale DCCT study in the USA (The Diabetes Control and Complications Trial Research Group (1993), N. Engl. J. Med. 329, 977-986) showed unambiguously that chronically elevated blood glucose levels are responsible for the development of late diabetic complications. Late diabetic complications are microvascular and macrovascular damage which is manifested in certain circumstances as retinopathy, nephropathy, or neuropathy, and leads to blindness, renal failure, and loss of extremities, and, in addition, is associated with an increased risk of cardiovascular disorders. From this it can be inferred that an improved therapy of diabetes must be aimed primarily at keeping blood glucose as closely as possible within the physiological range. According to the concept of intensified insulin therapy, this is to be achieved by means of injections, several times a day, of fast-acting and slow-acting insulin preparations. Fast-acting formulations are given at meal times, in order to compensate the postprandial rise in blood glucose. Slow-acting basal insulins are intended to ensure the basic supply of insulin, especially during the night, without leading to hypoglycemia.

Insulin is a polypeptide composed of 51 amino acids which are divided between two amino acid chains: the A chain, with 21 amino acids, and the B chain, with 30 amino acids. The chains are linked together by two disulfide bridges. Insulin preparations have been employed for many years in diabetes therapy. Such preparations use not only naturally occurring insulins but also, more recently, insulin derivatives and insulin analogs.

Insulin analogs are analogs of naturally occurring insulins, namely human insulin or animal insulins, which differ by replacement of at least one naturally occurring amino acid residue by other amino acids and/or by addition/deletion of at least one amino acid residue, from the corresponding, otherwise identical, naturally occurring insulin. The amino acids in question may also be amino acids which do not occur naturally.

Insulin derivatives are derivatives of naturally occurring insulin or an insulin analog which are obtained by chemical modification. The chemical modification may consist, for example, in the addition of one or more defined chemical groups to one or more amino acids. Generally speaking, the activity of insulin derivatives and insulin analogs is somewhat altered as compared with human insulin.

Insulin analogs with an accelerated onset of action are described in EP 0 214 826, EP 0 375 437, and EP 0 678 522. EP 0 124 826 relates, among other things, to replacements of B27 and B28. EP 0 678 522 describes insulin analogs which have different amino acids in position B29, preferably proline, but not glutamic acid. EP 0 375 437 encompasses insulin analogs with lysine or arginine at B28, which may also optionally be modified at B3 and/or A21.

EP 0 419 504 discloses insulin analogs which are protected from chemical modifications by modification of asparagine in B3 and of at least one further amino acid at positions A5, A15, A18 or A21.

Generally speaking, insulin derivatives and insulin analogs have a somewhat altered action as compared with human insulin.

WO 92/00321 describes insulin analogs in which at least one amino acid in positions B1-B6 has been replaced by lysine or arginine. Such insulins, according to WO 92/00321, have an extended effect. A delayed effect is also exhibited by the insulin analogs described in EP-A 0 368 187. The concept of intensified insulin therapy attempts to reduce the risk to health by aiming for stable control of the blood sugar level by means of early administration of basal insulins. One example of a common basal insulin is the drug Lantus® (active ingredient: insulin glargine=Gly (A21), Arg (B31), Arg (B32) human insulin). Generally speaking, the aim in the development of new, improved basal insulins is to minimize the number of hypoglycemic events. An ideal basal insulin acts safely in each patient for at least 24 hours. Ideally, the onset of the insulin effect is delayed and has a fairly flat time/activity profile, thereby significantly minimizing the risk of short-term undersupply of sugar, and allowing administration even without food being taken beforehand. The supply of basal insulin is effective when the insulin activity goes on consistently for as long as possible, i.e., the body is supplied with a constant amount of insulin. As a result, the risk of hypoglycemic events is low, and patient-specific and day-specific variability are minimized. The pharmacookinetic profile of an ideal basal insulin, then, ought to be characterized by a delayed onset of action and by a delayed action, i.e., a long-lasting and uniform action. The preparations of naturally occurring insulins for insulin replacement that are present on the market differ in the origin of the insulin (e.g., bovine, porcine, human insulin) and also in their composition, and so the activity profile (onset and duration of action) may be affected. Through combination of different insulin products it is possible to obtain any of a very wide variety of activity profiles and to bring about very largely physiological blood sugar values. Recombinant DNA technology nowadays allows the preparation of modified insulins of this kind. They include insulin glargine (Gly(A21)-Arg(B31)-Arg(B32) human insulin), with an extended duration of action. Insulin glargine is injected in the form of a clear, acidic solution, and, on the basis of its dissolution properties is precipitated, in the physiological pH range of the subcutaneous tissue, as a stable hexamer association. Insulin glargine is injected once a day and is notable in comparison with other long-active insulins for its flat serum profile and the associated reduction in the risk of night hypoglycemias (Schubert-Zsilavecz et al., 2:125-130 (2001)). In contrast to preparations described to date, the specific preparation of insulin glargine that leads to the prolonged duration of action is characterized by a clear solution with an acidic pH. Specifically at acidic pH, however, insulins exhibit reduced stability and an increased tendency toward aggregation under thermal and physico-mechanical load, which may be manifested in the form of haze and precipitation (particle formation) (Brange et al., J. Ph. Sci 86:517-525 (1997)).

In storage at 2-8° C., liquid insulin preparations have a shelf life of around 2 years. The shelf life in use allows storage at up to 25° C., and is given as being 4 weeks; mechanical stress (vibration) must be avoided. Patients, generally, must ensure that the insulin preparation remains a clear solution, since in exceptional cases there may be precipitation of the insulin, in part through formation of what are called “fibrils”. As a consequence of this, there is a risk that a sufficient dose of the drug will not be ensured.

There is therefore a need for the development of heat-stable and vibration-stable stable insulins, and heat-stable and vibration-stable stable insulin preparations, for the treatment of type 2 and type 1 diabetes. This has been accomplished as part of the invention described here. The 2-component insulin preparations of the invention differ from their conventional counterparts in their heat stability and in their stability toward mechanical stress. In contrast to liquid insulin formulations available on the market, the heat stability and vibration stability is based on the preservation of insulin in solid form until a short time before administration. It has been possible to show that solid insulin is more stable to degradation (change in molecular structure) under heat stress than dissolved insulin. Moreover, dissolved insulin undergoes precipitation; this constitutes a biophysical process which cannot take place in the solid state. Consequently, with comparable heat stress, there is more bioavailable insulin available from an insulin preparation if the dissolution process takes place after the heat stress.

Aqueous insulin preparations, moreover, show a tendency to precipitate insulin under mechanical stress (vibration). The amount of bioavailable insulin after vibration stress is therefore unknown and constitutes a detraction from patient safety. Moreover, apart from heat-stable insulin preparations, there is a need for those preparations which are also stable with respect to mechanical stress (e.g., vibration). It has been possible to show that solid insulin is more stable to vibration than dissolved insulin. The biophysical process of precipitation on vibration stress occurs exclusively in dissolved insulin. Consequently, with comparable vibration stress, bioavailable insulin is available in an unaffected amount if the dissolution process takes place after the vibration stress.

Insulins are typically available on the market in aqueous systems which comprise auxiliaries and adjuvants such as, for example, antibactericides, isotonicity agents, buffer substances, and/or surfactants (=“solvents” below). The dissolution of insulins for the purpose of preparing pharmacological preparations is generally ensured by the insulins first being acidically dissolved, before then being adjusted, in further steps, to the desired concentration and pH of the solution (The Wellcome Foundation Limited to London, Octrooiraad Nederland, Octrooiaanvrage No. 6506714, May 26, 1965 “Werkwijze voor het bereiden van insulinepreparaten”).

Described alternatively is the dissolution of insulins in the alkaline range with subsequent adjustment of pH, which is associated with a slightly increased stability on the part of the insulin preparations (WO 2004/096266).

In each case, therefore, a multistage operation is necessary in order to prepare a ready-to-use insulin preparation, encompassing, among other stages, the adjustment of the pH.

It has now surprisingly been found that ready-to-use insulin preparations are preparable by a single-stage method in which the insulin (or an analog or derivative thereof) is combined with a buffer solution, and dissolving the insulin (or the analog or derivative thereof) produces, within a few minutes, the ready-to-use insulin preparation.

Consequently the process of dissolving the solid insulin can take place in situ by the patient or his or her carer, in a two-chamber ampoule or other suitable device, for example. Corresponding attempts to dissolve insulins completely in the suitable solvents and concentrations within a reasonable time (˜10 min) have run successfully. The success achieved in this is dependent substantially on the suitable pH of the solvent. The fundamental composition of the solvents (auxiliaries, additives, etc.) is no different, or not necessarily different, from that of formulations which are already on the market.

Insulins stable to heat and to mechanical stress therefore provide the following advantages:
no need for cooling chain
simpler keeping of stocks in hot countries
improvement in drug safety for patient
general extended drug shelf life
reduced return of ampoules containing precipitated insulin.

The 2-component insulin preparations of the invention afford the advantages identified above. In this way, patients with no access or with imperfect access to suitable cooling equipment are able to hold stocks of insulin; the use of such preparations in countries with a hot climate is particularly advantageous as well.

By means of suitable ampoule sizes in the sense of the 2-component insulin preparations of the invention, it is also possible to reduce the amount of insulin solution for the patient in such a way that the required period of storage in use can be reduced to a minimum. This allows the addition of antimicrobial adjuvants such as m-cresol or other phenols, for example, to be reduced or done away with entirely.

The preparations can be prepared for all known insulins, insulin analogs, and insulin derivatives.

This includes preparations having desired basal time/activity profiles, in which the insulin analogs are characterized by the features that
the B chain end is composed of an amidated basic amino acid residue such as lysine or arginine amide, i.e., in the amidated basic amino acid residue at the B chain end, the carboxyl group of the terminal amino acid is in its amidated form, and
the N-terminal amino acid residue of the insulin A chain is a lysine or arginine residue, and
the amino acid position A8 is occupied by a histidine residue, and
the amino acid position A21 is occupied by a glycine residue, and
there are two replacements of neutral amino acids by acidic amino acids, two additions of negatively charged amino acid residues, or one such replacement and one such addition, in each of positions A5, A15, A18, B-1, B0, B1, B2, B3, and B4.

The invention accordingly provides a method for preparing an aqueous, pharmaceutical formulation comprising an insulin, insulin analog or insulin derivative, or a pharmacologically tolerable salt thereof, the ready-to-use formulation being effected directly by dissolving the insulin, insulin analog or insulin derivative in solid form with a suitable solvent mixture, preferably in which the composition of the suitable solvent mixture is determined by
(a) preparing solvent mixtures differing in pH and having excipient concentrations corresponding to the final excipient concentration of the formulation comprising an insulin, insulin analog or insulin derivative, and
(b) by dissolving the desired insulin, insulin analog or insulin derivative, determining that solvent mixture which, following dissolution of the solid of insulin, insulin analog or insulin derivative, produces the desired pH of the ready-to-use formulation.

The invention further provides a method as described above, the insulin, insulin analog or insulin derivative taking the form of a crystalline or amorphous solid.

The invention provides a method as described above, the insulin being selected from a group containing human insulin, porcine insulin, and bovine insulin.

The invention further provides a method as described above, the insulin analog being selected from the group containing Gly(A21), Arg(B31), Arg(B32) human insulin, Lys(B3), Glu(B29) human insulin, Asp(B28) human insulin, Lys(B28) Pro(B29) human insulin, and Des(B30) human insulin.

The invention further provides a method as claimed in one or more of claims 1 to 3, the insulin analog being selected from the group containing an insulin analog of the formula I

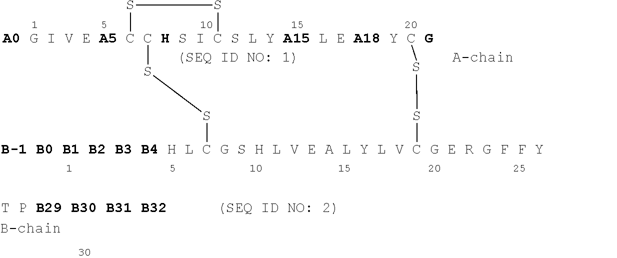

where
A0 is Lys or Arg;
A5 is Asp, Gln or Glu;
A15 is Asp, Glu or Gln;
A18 is Asp, Glu or Asn;
B-1 is Asp, Glu or an amino group;
B0 is Asp, Glu or a chemical bond;
B1 is Asp, Glu or Phe;
B2 is Asp, Glu or Val;
B3 is Asp, Glu or Asn;
B4 is Asp, Glu or Gln;
B29 is Lys or a chemical bond;
B30 is Thr or a chemical bond;
B31 is Arg, Lys or a chemical bond;
B32 is Arg-amide, Lys-amide or an amino group,
   where two amino acid residues of the group containing A5, A15, A18, B-1, B0, B1, B2, B3, and B4, simultaneously and independently of one another, are Asp or Glu, or a pharmacologically tolerable salt thereof, preferably in which the insulin analog is selected from a group containing:
Arg (A0), His (A8), Glu (A5), Asp (A18), Gly (A21), Arg (B31), Arg (B32)-NH₂ human insulin,
Arg (A0), His (A8), Glu (A5), Asp (A18), Gly (A21), Arg (B31), Lys (B32)-NH₂ human insulin,
Arg (A0), His (A8), Glu (A15), Asp (A18), Gly (A21), Arg (B31), Arg (B32)-NH₂ human insulin,
Arg (A0), His (A8), Glu (A15), Asp (A18), Gly (A21), Arg (B31), Lys (B32)-NH₂ human insulin,
Arg (A0), His (A8), Glu(A5), Glu (A15), Gly (A21), Arg (B31), Arg (B32)-NH₂ human insulin,
Arg (A0), His (A8), Glu (A5), Glu (A15), Gly (A21), Arg (B31), Lys (B32)-NH₂ human insulin,
Arg (A0), His (A8), Glu (A5), Gly (A21), Asp (B3), Arg (B31), Arg (B32)-NH₂ human insulin,
Arg (A0), His (A8), Glu (A5), Gly (A21), Asp (B3), Arg (B31), Lys (B32)-NH₂ human insulin,
Arg (A0), His (A8), Glu (A15), Gly (A21), Asp (B3), Arg (B31), Arg (B32)-NH₂ human insulin,
Arg (A0), His (A8), Glu (A15), Gly (A21), Asp (B3), Arg (B31), Lys (B32)-NH₂ human insulin,
Arg (A0), His (A8), Asp (A18), Gly (A21), Asp (B3), Arg (B31), Arg (B32)-NH₂ human insulin,
Arg (A0), His (A8), Asp (A18), Gly (A21), Asp (B3), Arg (B31), Lys (B32)-NH₂ human insulin,
Arg (A0), His (A8), Gly (A21), Asp (B3), Glu (B4), Arg (B31), Arg (B32)-NH₂ human insulin,
Arg (A0), His (A8), Gly (A21), Asp (B3), Glu (B4), Arg (B31), Lys (B32)-NH₂ human insulin,
Arg (A0), His (A8), Glu (A5), Gly (A21), Glu (B4), Arg (B31), Arg (B32)-NH₂ human insulin,
Arg (A0), His (A8), Glu (A5), Gly (A21), Glu (B4), Arg (B31), Lys (B32)-NH₂ human insulin,
Arg (A0), His (A8), Glu (A15), Gly (A21), Glu (B4), Arg (B31), Arg (B32)-NH₂ human insulin,
Arg (A0), His (A8), Glu (A15), Gly (A21), Glu (B4), Arg (B31), Lys (B32)-NH₂ human insulin,
Arg (A0), His (A8), Asp (A18), Gly (A21), Glu (B4), Arg (B31), Arg (B32)-NH₂ human insulin,
Arg (A0), His (A8), Asp (A18), Gly (A21), Glu (B4), Arg (B31), Lys (B32)-NH₂ human insulin,
Arg (A0), His (A8), Glu (A5), Gly (A21), Glu (B0), Arg (B31), Arg (B32)-NH₂ human insulin,
Arg (A0), His (A8), Glu (A5), Gly (A21), Glu (B0), Arg (B31), Lys (B32)-NH₂ human insulin,
Arg (A0), His (A8), Glu (A15), Gly (A21), Glu (B0), Arg (B31), Arg (B32)-NH₂ human insulin,
Arg (A0), His (A8), Glu (A15), Gly (A21), Glu (B0), Arg (B31), Lys (B32)-NH₂ human insulin,
Arg (A0), His (A8), Asp (A18), Gly (A21), Glu (B0), Arg (B31), Arg (B32)-NH₂ human insulin,
Arg (A0), His (A8), Asp (A18), Gly (A21), Glu (B0), Arg (B31), Lys (B32)-NH₂ human insulin,
Arg (A0), His (A8), Glu (A5), Gly (A21), Asp (B1), Arg (B31), Arg (B32)-NH₂ human insulin,
Arg (A0), His (A8), Glu (A5), Gly (A21), Asp (B1), Arg (B31), Lys (B32)-NH₂ human insulin,
Arg (A0), His (A8), Glu (A15), Gly (A21), Asp (B1), Arg (B31), Arg(B32)-NH₂ human insulin,
Arg (A0), His (A8), Glu (A15), Gly (A21), Asp (B1), Arg (B31), Lys (B32)-NH₂ human insulin,
Arg (A0), His (A8), Asp (A18), Gly (A21), Asp (B1), Arg (B31), Arg (B32)-NH₂ human insulin,
Arg (A0), His (A8), Asp (A18), Gly (A21), Asp (B1), Arg (B31), Lys (B32)-NH₂ human insulin,
(A0), His (A8), Gly (A21), Glu (B0), Asp (B1), Arg (B31), Arg (B32)-NH₂ human insulin,
Arg (A0), His (A8), Gly (A21), Glu (B0), Asp (B1), Arg (B31), Lys (B32)-NH₂ human insulin,
Arg (A0), His (A8), Asp (A18), Gly (A21), Asp (B3), Arg (B30), Arg (B31)-NH₂ human insulin,
Arg (A0), His (A8), Asp (A18), Gly (A21), Asp (B3), Arg (B30), Lys (B31)-NH₂ human insulin.

The invention further provides a method as described above, the insulin analog being selected from the containing

an insulin analog of the formula II

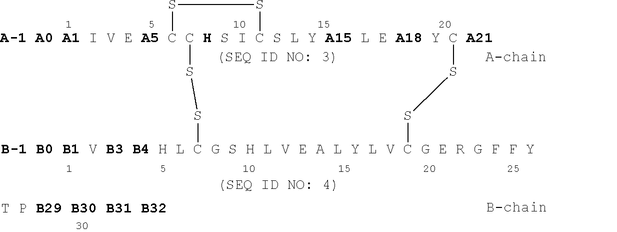

where
A-1 is Lys, Arg or an amino group;
A0 is Lys, Arg or a chemical bond;
A1 is Arg or Gly;
A5 is Asp, Glu or Gln;
A15 is Asp, Glu or Gln;
A18 is Asp, Glu or Asn;
A21 is Ala, Ser, Thr or Gly;
B-1 is Asp, Glu or an amino group;
B0 is Asp, Glu or a chemical bond;
B1 is Asp, Glu, Phe or a chemical bond;
B3 is Asp, Glu or Asn;
B4 is Asp, Glu or Gln;
B29 is Arg, Lys or an amino acid selected from the group containing the amino acids Phe, Ala, Thr, Ser, Val, Leu, Glu or Asp, or a chemical bond;
B30 is Thr or a chemical bond;
B31 is Arg, Lys or a chemical bond;
B32 is Arg-amide or Lys-amide,
   where not more than one amino acid residue from the group containing A5, A15, A18, B-1, B0, B1, B2, B3 and B4, simultaneously and independently of one another, is Asp or Glu, preferably in which the insulin analog is selected from a group containing:
Arg (A-1), Arg (A0), Glu (A5), His (A8), Gly (A21), Arg (B30)-NH₂ human insulin,
Arg (A-1), Arg (A0), Glu (A5), His (A8), Gly (A21), Lys (B30)-NH₂ human insulin,
Arg (A-1), Arg (A0), Glu (A15), His (A8), Gly (A21), Arg (B30)-NH₂ human insulin,
Arg (A-1), Arg (A0), Glu (A15), His (A8), Gly (A21), Lys (B30)-NH₂ human insulin,
Arg (A-1), Arg (A0), Asp (A18), His (A8), Gly (A21), Arg (B30)-NH₂ human insulin,
Arg (A-1), Arg (A0), Asp (A18), His (A8), Gly (A21), Arg (B30)-NH₂ human insulin,
Arg (A-1), Arg (A0), His (A8), Gly (A21), Glu (B0), Arg (B30)-NH₂ human insulin,
Arg (A-1), Arg (A0), His (A8), Gly (A21), Glu (B0), Lys (B30)-NH₂ human insulin,
Arg (A-1), Arg (A0), His (A8), Gly (A21), Asp (B3), Arg (B30)-NH₂ human insulin,
Arg (A-1), Arg (A0), His (A8), Gly (A21), Asp (B3), Lys (B30)-NH₂ human insulin,
Arg (A-1), Arg (A0), His (A8), Gly (A21), Glu (B4), Arg (B30)-NH₂ human insulin,
Arg (A-1), Arg (A0), His (A8), Gly (A21), Glu (B4), Lys (B30)-NH₂ human insulin,
Arg (A0), His (A8), Gly (A21), Arg (B31), Arg (B32)-NH₂ human insulin,
Arg (A0), His (A8), Gly (A21), Arg (B31), Lys (B32)-NH₂ human insulin,
Arg (A0), Glu (A5), His (A8), Gly (A21), Arg (B31), Arg (B32)-NH₂ human insulin,
Arg (A0), Glu (A5), His (A8), Gly (A21), Arg (B31), Lys (B32)-NH₂ human insulin,
Arg (A0), Asp (A18), His (A8), Gly (A21), Arg (B31), Arg (B32)-NH₂ human insulin,
Arg (A0), Asp (A18), His (A8), Gly (A21), Arg (B31), Lys (B32)-NH₂ human insulin,
Arg (A0), Glu (A15), His (A8), Gly (A21), Arg (B31), Arg (B32)-NH₂ human insulin,
Arg (A0), Glu (A15), His (A8), Gly (A21), Arg (B31), Lys (B32)-NH₂ human insulin,
Arg (A0), His (A8), Gly (A21), Asp (B3), Arg (B31), Arg (B32)-NH₂ human insulin,
Arg (A0), His (A8), Gly (A21), Asp (B3), Arg (B31), Lys (B32)-NH₂ human insulin,
Arg (A0), His (A8), Gly (A21), Glu (B4), Arg (B31), Arg (B32)-NH₂ human insulin,
Arg (A0), His (A8), Gly (A21), Glu (B4), Arg (B31), Lys (B32)-NH₂ human insulin,
Arg (A0), His (A8), Gly (A21), Glu (B0), Arg (B31), Arg (B32)-NH₂ human insulin,
Arg (A0), His (A8), Gly (A21), Glu (B0), Arg (B31), Lys (B32)-NH₂ human insulin,
Arg (A0), His (A8), Gly (A21), Arg (B30)-NH₂ human insulin,
Arg (A0), His (A8), Gly (A21), Lys (B30)-NH₂ human insulin,
Arg (A-1), Arg (A0), His (A8), Gly (A21), Arg (B30)-NH₂ human insulin,
Arg (A-1), Arg (A0), His (A8), Gly (A21), Lys (B30)-NH₂ human insulin,
Arg (A0), Arg (A1), His (A8), Gly (A21), Arg (B30)-NH₂ human insulin,
Arg (A0), Arg (A1), His (A8), Gly (A21), Lys (B30)-NH₂ human insulin,
His (A8), Gly (A21), Arg (B31), Arg (B32)-NH₂ human insulin.

The invention further provides a method as claimed in one or more of claims 1 to 3, the insulin derivative being selected from the group containing B29-N-myristoyl-des(B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28}Pro^{B29} human insulin, B30-N-myristoyl-Thr^{B29}Lys^{B30} human insulin, B30-N-palmitoyl-Thr^{B29}Lys^{B30} human insulin, B29-N-(N-palmitoyl-γ-glutamyl)-des(B39) human insulin, B29-N-(N-lithocholyl-γ-glutamyl)-des(B30) human insulin, B29-N-(ω-carboxyheptadecanoyl)-des(B30) human insulin and B29-N-(ω-carboxyheptadecanoyl) human insulin.

The invention further provides a method as described above, the formulation comprising a preservative selected from a group containing phenol, m-cresol, chlorocresol, benzyl alcohol, and parabens.

The invention further provides a method as described above, the formulation comprising an isotonicity agent selected from a group containing mannitol, sorbitol, lactose, dextrose, trehalose, sodium chloride, and glycerol.

The invention further provides a method as described above, the insulin, insulin analog and/or insulin derivative being present in a concentration of 240-3000 nmol/ml.

The invention further provides a method as described above, the formulation further comprising a glucagon-like peptide-1 (GLP1) or an analog or derivative thereof, or exendin-3 and/or -4 or an analog or derivative thereof, preferably in which an analog of exendin-4 is selected from a group containing
H-desPro³⁶-exendin-4-Lysₛ-NH₂,
H-des(Pro^{36,37})-exendin-4-Lys₄-NH₂ and
H-des(Pro^{36,37})-exendin-4-Lys₅-NH₂,
   or a pharmacologically tolerable salt thereof.

The invention further provides a method as described above, in which an analog of exendin-4 is selected from the group containing
desPro³⁶ [Asp²⁸]exendin-4 (1-39),
desPro³⁶ [IsoAsp²⁸]exendin-4 (1-39),
desPro³⁶ [Met(O)¹⁴, Asp²⁸]exendin-4 (1-39),
desPro³⁶ [Met(O)¹⁴, IsoAsp²⁸]exendin-4 (1-39),
desPro³⁶ [Trp(O₂)²⁵, Asp²⁸]exendin-2 (1-39),
desPro³⁶ [Trp(O₂)²⁵, IsoAsp²⁸]exendin-2 (1-39),
desPro³⁶ [Met(O)¹⁴Trp(O₂)²⁵, Asp²⁸]exendin-4 (1-39) and
desPro³⁶ [Met(O)¹⁴Trp(O₂)²⁵, IsoAsp²⁸]exendin-4 (1-39),
   or a pharmacologically tolerable salt thereof, preferably in which the peptide Lys₆-NH₂ is attached to the C-termini of the analogs of exendin-4.

The invention further provides a pharmaceutical formulation as described above, in which an analog of exendin-4 is selected from the group containing
H-(Lys)₆-des Pro³⁶ [Asp²⁸]exendin-4(1-39)-Lys₆-NH₂
des Asp²⁸Pro³⁶, Pro³⁷, Pro³⁸ exendin-4(1-39)-NH₂,
H-(Lys)₆-des Pro³⁶, Pro³⁷, Pro³⁸ [Asp²⁸]exendin-4(1-39)-NH₂,
H-Asn-(Glu)₅ des Pro³⁶, Pro³⁷, Pro³⁸ [Asp²⁸]exendin-4(1-39)-NH₂,
des Pro³⁶, Pro³⁷, Pro³⁸ [Asp²⁸]exendin-4(1-39)-(Lys)₆-NH₂
H-(Lys)₆-des Pro³⁶, Pro³⁷, Pro³⁸ [Asp²⁸]exendin-4(1-39)-(Lys)₆-NH₂,
H-Asn-(Glu)₅-des Pro³⁶, Pro³⁷, Pro³⁸ [Asp²⁸]exendin-4(1-39)-(Lys)₆-NH₂,
H-(Lys)₆-des Pro³⁶ [Trp(O₂)²⁵, Asp²⁸]exendin-4(1-39)-Lys₆-NH₂,
H-des Asp²⁸ Pro³⁶, Pro³⁷, Pro³⁸ [Trp(O₂)²⁵]exendin-4(1-39)-NH₂,
H-(Lys)₆-des Pro³⁶, Pro³⁷, Pro³⁸ [Trp(O₂)²⁵, Asp²⁸]exendin-4(1-39)-NH₂,
H-Asn-(Glu)₅-des Pro³⁶, Pro³⁷, Pro³⁸ [Trp(O₂)²⁵, Asp²⁸]exendin-4(1-39)-NH₂,
des Pro³⁶, Pro³⁷, Pro³⁸ [Trp(O₂)²⁵, Asp²⁸]exendin-4(1-39)-(Lys)₆-NH₂,
H-(Lys)₆-des Pro³⁶, Pro³⁷, Pro³⁸ [Trp(O₂)²⁵, Asp²⁸]exendin-4(1-39)-(Lys)₆-NH₂,
H-Asn-(Glu)₅-des Pro³⁶, Pro³⁷, Pro³⁸ [Trp(O₂)²⁵, Asp²⁸]exendin-4(1-39)-(Lys)₆-NH₂,
H-(Lys)₆-des Pro³⁶ [Met(O)¹⁴, Asp²⁸]exendin-4(1-39)-Lys₆-NH₂,
des Met(O)¹⁴ Asp²⁸ Pro³⁶, Pro³⁷, Pro³⁸ exendin-4(1-39)-NH₂,
H-(Lys)₆-des Pro³⁶, Pro³⁷, Pro³⁸ [Met(O)¹⁴, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Pro³⁷, Pro³⁸ [Met(O)¹⁴, Asp²⁸]exendin-4(1-39)-(Lys)₆-NH₂,
H-(Lys)₆-des Pro³⁶, Pro³⁷, Pro³⁸ [Met(O)¹⁴, Asp²⁸]exendin-4(1-39)-Lys₆-NH₂
H-Asn-(Glu)₅ des Pro³⁶, Pro³⁷, Pro³⁸ [Met(O)¹⁴, Asp²⁸]exendin-4(1-39)-(Lys)₆-NH₂,
H-(Lys)₆-des Pro³⁶ [Met(O)¹⁴, Trp(O₂)²⁵, Asp²⁸]exendin-4(1-39)-Lys₆-NH₂,
des Asp²⁸ Pro³⁶, Pro³⁷, Pro³⁸ [Met(O)¹⁴, Trp(O₂)²⁵]exendin-4(1-39)-NH₂,
H-(Lys)₆-des Pro³⁶, Pro³⁷, Pro³⁸ [Met(O)¹⁴, Trp(O₂)²⁵, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Pro³⁷, Pro³⁸ [Met(O)¹⁴, Trp(O₂)²⁵, Asp²⁸]exendin-4(1-39)-(Lys)₆-NH₂,
H-(Lys)₆-des Pro³⁶, Pro³⁷, Pro³⁸ [Met(O)¹⁴, Trp(O₂)²⁵, Asp²⁸]exendin-4(1-39)-(Lys)₆-NH₂,
H-Asn-(Glu)₅-des Pro³⁶, Pro³⁷, Pro³⁸ [Met(O)¹⁴, Trp(O₂)²⁵, Asp²⁸]exendin-4(1-39)-(Lys)₆-NH₂,
   or a pharmacologically tolerable salt thereof.

The invention further provides a method as described above, the formulation further comprising Arg³⁴, Lys²⁶ (N^{ε}(γ-glutamyl(N^{α}-hexadecanoyl))) GLP-1 (7-37) [liraglutide] or a pharmacologically tolerable salt thereof.

The invention further provides a method as described above, the formulation further comprising a zinc salt.

The invention further provides for the use of a method as described above in the industrial preparation of an insulin, insulin analog or insulin derivative.

The invention additionally provides a two-chamber injection system, in which one chamber contains an insulin, insulin analog or an insulin derivative in solid form and the other chamber

contains a solvent mixture having a defined pH and having the final excipient concentration of a desired formulation of an insulin, insulin analog or insulin derivative, for heat-stable and vibration-stable preservation of the insulin, insulin analog or insulin derivative for the later preparation of a ready-to-use formulation by dissolving of the solid in the solvent mixture as described above.

In this description, the terms “formulation” and “preparation” are used synonymously.

### KEY TO FIGURES

FIG. 1: blood sugar reducing effect of new insulin analogs of formula I in rats

FIG. 2: blood sugar reducing effect of new insulin analogs of formula I in dogs

FIG. 3: blood sugar reducing effect of YKL205 in dogs

FIG. 4: zinc dependence of hypoglycemic effect of YKL205 in dogs

FIG. 5: blood sugar reducing effect of inventive insulin analogs of formula II in rats

FIG. 6: blood sugar reducing effect of insulin glargine in rats

### EXAMPLES

The examples below are intended to illustrate the concept of the invention, without having any restricting effect.

### Example 1

### Simplified Dissolution of Insulins in One Step (Dissolution Test)

Insulins are typically available on the market in aqueous systems which comprise auxiliaries and adjuvants such as, for example, antibactericides, isotonicity agents, buffer substances, and/or surfactants (=“solvents” below). The dissolution of insulins for the purpose of preparing pharmacological preparations is generally ensured by the insulins first being acidically dissolved, before then being adjusted, in further steps, to the desired concentration and pH of the solution (The Wellcome Foundation Limited to London, Octrooiraad Nederland, Octrooiaanvrage No. 6506714, May 26, 1965 “Werkwijze voor het bereiden insulinepreparaten”).

Alternatively, the dissolution of insulins in the alkaline range is described, associated with a slightly elevated stability on the part of the insulin preparations (WO 2004/096266 PCT/DK2004/000300, “Improved Physical Stability of Insulin Formulations”).

### Dissolution Test:

In order to avoid the complicated procedure of the dissolution of insulins (see above), tests were carried out to determine whether insulins in solid form can be dissolved in such a way as to obtain their formulation, as present on the market, in one step. The objective was to retain the composition of the market formulation, in other words including the concentration of all auxiliaries and additives, and the pH.

The following were investigated:
a. Lantus® (auxiliaries and additives zinc chloride, m-cresol, glycerol, pH4)
b. Insuman® (auxiliaries and additives sodium hydrogen phosphate, m-cresol, glycerol, pH 7.3)

Different solvent mixtures were prepared, corresponding to the final excipients concentration for the two insulins Lantus® and Insuman®. The pH of the solvent was varied.

a. Lantus® required a solvent pH of 2.85 in order to achieve complete dissolution with the required final pH value of pH 4. The dissolution rate (final volume 1 ml) was below 10 min.

b. Insuman® required a solvent pH of 7.6 in order to achieve complete dissolution with the required final pH value of pH 7.3. The dissolution rate (final volume 1 ml) was below 10 min.

LC-UV/MS analysis of the samples following the simplified dissolution with subsequent evaluation of the UV chromatogram gave spectra which were identical with the market formulations (UV signal of the m-cresol and of the respective insulin in relation to retention time and peak area).

Conclusion: it is possible to dissolve insulins in one step, i.e., without prior dissolution at acidic or alkaline pH. The key factor identified was the adaptation of the pH of the solvent to the particular target concentration, and also the pH of the completed product as a function of the intrinsic, pH-modifying properties of the insulins to be dissolved. With adaptation of these empirically determined parameters, it was possible to accomplish the dissolution procedure for the insulins within an appropriate time frame (<10 min).

### Example 2

### Heat Stability of Solids (Heat Stress Test, Vibration Test, Amorphous Solids)

The heat stability of aqueous insulin preparations has been a subject of numerous investigations (reference: A. Krogh, A. M. Hemmingsen, Biochem. J. (1928) 22, 1231-1238 “The destructive action of heat on insulin solutions”).

It is known that additives have different contributions to the thermal stability of the dissolved insulin, examples being the zinc concentration or the pH (N. R. Stephenson, R. G. Romans; Journal of Pharmacy and Pharmacology (1960), 12 372-376 “Thermal Stability of Insulin made from Zinc Insulin Crystals”).

In the investigation of heat stability of in-house, market-ready solutions, containing all of the auxiliaries and additives, our own studies as well identified precipitation—as well as the formation of degradation products—as the principal cause of the loss of biologically active insulin material (B. V. Fisher, P. B. Porter; J. Pharm. Pharmacol. (1981), 33 203-206 “Stability of bovine Insulin”).

On the basis of these observations, the heat stability of solid insulin was examined with the trade names that are used of the ready-to-use aqueous formulations also having been used for the respective solid insulins (active ingredients):

### Heat Stress Test:

I) Different solid insulins were stored for two weeks at different temperatures (50° C., 60° C. and 80° C.) (absence of light).
   a. Lantus® (crystalline); b. Insuman® (crystalline); c. Apidra® (amorphous)
II) Different solid insulins were stored at 60° C. for different times (14 days, 1 month, 2 months, 3 months).
   a. Lantus® (crystalline); b. Insuman® (crystalline); c. Apidra® (amorphous)
III) Solids of Arg (A0), His (A8), Glu (A15), Asp (A18), Gly (A21), Arg (B31), Arg (B32) human insulin amide and Lantus®, both in amorphous state, were stored for two weeks at 60° C.

After the end of the heat stress phases, the solids were dissolved, producing a concentration identical to that of the market formulations.

As comparative examples, insulin solutions with the same concentration were dissolved directly prior to measurement.

In addition, Lantus®, Insuman® and Apidra® in market-ready preparations/containers (source: production) were stressed at 60° C. for 14 days. As comparative examples, containers from the same production batch were stored at 4° C. and analyzed together with the stressed samples.

Conclusion: the heat stability of insulins on storage in solid form for 14 days proved to be temperature-dependent. The degree of stability was insulin-dependent. Storage in solid form proved to be more heat stable than storage as a market-ready solution.

(I) LC-UV/MS analysis of the samples with subsequent evaluation of UV chromatogram gave the following results:

| Lantus ®-solid | | | |
|---|---|---|---|
| 14 days | 50° C. | 60° C. | 80° C. |
| Remaining Lantus ® | ~100% | ~100% | ~90% |
| By-products | ~0% | ~0% | ~4% |
| Haze | no | no | no |

| | 14 days | 14 days | |
|---|---|---|---|
| Lantus ®-solution | cartridge | vial without | 14 days |
| 14 days @ 60° | without PS20 | PS20 | vial with PS20 |
| Remaining Lantus ® | ~65% | ~30% | ~80% |
| By-products | ~15% | ~5% | ~20% |
| Haze | yes | severe | none |

| Insuman ®-solid | | | |
|---|---|---|---|
| 14 days | 50° C. | 60° C. | 80° C. |
| Remaining Insuman ® | ~90% | ~85% | ~65% |
| By-products | ~4% | ~10% | ~25% |
| Haze | no | no | no |

| Insuman ®-solution | | |
|---|---|---|
| 14 days @ 60° | Cartridge | Vial |
| Remaining Insuman ® | ~1% | ~7% |
| By-products | ~10% | ~35% |
| Haze | severe | severe |

| Apidra ®-solid | | | |
|---|---|---|---|
| 14 days | 50° C. | 60° C. | 80° C. |
| Remaining Apidra ® | ~80% | ~80% | ~55% |
| By-products | ~2% | ~3% | ~7% |
| Haze | no | no | no |

| Apidra ®-solution | | |
|---|---|---|
| 14 days @ 60° | Cartridge | Vial |
| Remaining Apidra ® | ~1% | ~1% |
| By-products | ~5% | ~10% |

Conclusion: the heat stability of insulins on storage in solid form for 14 days proved to be temperature-dependent. The degree of stability was insulin-dependent. Storage in solid form proved to be more heat-stable than storage as a market-ready solution.

(II) LC-UV/MS analysis of the samples with subsequent evaluation of UV chromatogram gave the following results:

Lantus ®:

| Lantus ®-solid | | | | | |
|---|---|---|---|---|---|
| at 60° | 14 days | 1 month | 2 months | 3 months | 4 months |
| Remaining | ~100% | ~99% | ~95% | ~95% | ~95% |
| Lantus ® | | | | | |
| By-products | ~0% | ~1% | ~5% | ~5% | ~5% |
| Haze | no | no | no | no | no |

Insuman ®:

| Insuman ®-solid | | | | | |
|---|---|---|---|---|---|
| at 60° | 14 days | 1 month | 2 months | 3 months | 4 months |
| Remaining | ~85% | ~70% | ~75% | ~70% | ~70% |
| Insuman ® | | | | | |
| By-products | ~10% | ~20% | ~20% | ~20% | ~20% |
| Haze | No | no | no | no | no |

Apidra ®:

| Apidra ®-solid | | | | | |
|---|---|---|---|---|---|
| at 60° | 14 days | 1 month | 2 months | 3 months | 4 months |
| Remaining | ~80% | n.a. | ~65% | ~70% | ~70% |
| Apidra ® | | | | | |
| By-products | ~2% | n.a. | ~10% | ~10% | ~10% |

Conclusion: the heat stability of insulins on storage in solid form at 60° C. proved to be time-dependent. The degree of stability was insulin-dependent.

(III) LC-UV/MS analysis of the samples with subsequent evaluation of the UV chromatogram gave the following results:

| | | Arg (A0), His (A8), |
|---|---|---|
| | | Glu (A15), Asp (A18), |
| Amorphous solid 14 | | Gly (A21), Arg (B31), |
| days at 60° C. | Lantus ® | Arg (B32) human insulin amide |
| Remaining Insulin | ~100% | ~90% |
| By-products | ~1% | ~3% |
| Haze | No | no |

Conclusion: the heat stability of insulins on storage in solid form at 60° C. proved to be time-dependent. The degree of stability was insulin-dependent. Both of the long-acting insulins investigated proved to be relatively heat-stable in the amorphous state as well.

### Vibration Test:

It is known that aqueous insulin preparations are unstable with respect to vibration (A. Oliva, J. B. Farina, M. Llabres; Int. J. Of Pharmaceutics (1996), 143 143-170 “Influence of temperature and shaking on stability of insulin preparations: degradation kinetics”).

Different insulin formulations were vibrated at 1400 rpm at room temperature (absence of light) for 100 h.
a) Lantus®, Insuman® and Apidra® in market-ready preparations/containers (source: production)
   Lantus (cartridge without Polysorbate 20, vial without Polysorbate 20, vial with Polysorbate 20), Insuman® (cartridge, vial), Apidra® (cartridge, vial)
b) Lantus®, Insuman® and Apidra® in solid form (vibrated separately: solvent for direct dissolution in one step; see “Direct dissolution of solid insulin”).

After the end of the vibration phase, the solids (samples b) were dissolved, producing a concentration identical to that of the market formulations.
c) As comparative samples, the corresponding market formulations were also stored at room temperature in the absence of light for 100 h.

LC-UV/MS analysis of the samples with subsequent evaluation of the UV chromatogram gave the following results (reference: samples c):

| | without | | | |
|---|---|---|---|---|
| | PS20, | without PS 20, | with PS 20, | Solid/solution |
| Lantus ® | cartridge | vial | vial | separately |
| Remaining | ~85% | ~82% | ~95% | ~100% |
| Lantus ® | | | | |
| By-products | none | none | none | none |
| Haze | yes | yes | little | no |

| Insuman ® | Cartridge | Vial | Solid/solution separately |
|---|---|---|---|
| Remaining | ~0% | ~0% | ~100% |
| Insuman ® | | | |
| By-products | none | none | none |
| Haze | severe | severe | no |

| Apidra ® | Cartridge | Vial | Solid/solution separately |
|---|---|---|---|
| Remaining | ~98% | ~77% | ~100% |
| Insuman ® | | | |
| By-products | none | none | none |
| Haze | little | yes | no |

Conclusion: mechanical stress by vibration on insulin solutions caused precipitation of insulin. The extent is insulin-dependent: Insuman® showed the greatest instability here. Mechanical stress did not show any adverse consequences on solid insulin; the solid could be dissolved to an extent of 100% subsequently, even when the solutions as well were vibrated separately.

### Example 3

### Possible Applications (Industrial Manufacture of Insulin Preparations, Heat-Stable and Vibration-Stable Administration Form in a Two-Component System)

### Industrial Manufacture Insulin Preparations:

Dissolution in one step is a simplified process with the following advantages: there are fewer steps, with fewer intermediate analyses (e.g., pH) to be taken, i.e., the manufacture of the insulin preparations can take place more quickly. Moreover, the manufacture is less complicated, entailing simplified training of employees (less complicated SOPs). In addition there are fewer containers to be cleaned, achieving savings in turn in work time and material resources.

Heat-stable and vibration-stable administration form in a two-component system:

It has been found that solid insulins—even over a prolonged period of time—are more stable than dissolved insulins under heat stress. While there were instances of degradation to a minor extent, there was no precipitation loss, and so, for comparable heat stress, more bioavailable insulin was available when the dissolution procedure took place after the heat stress. Precipitation and degradation, induced by strong vibration of the solid insulins, was not observed.

Administration in solid form can therefore be described as being generally more temperature-stable and also more robust with respect to vibration, and, consequently, as safer. Furthermore, administering the insulins in solid form (powder) affords the additional advantage that accidental freezing by the patient cannot result in any complication; solid insulins are stored, as is known, in the frozen state.

Possible application: insulin powders instead of dissolved insulins are offered with suitable solvent (generally aqueous systems comprising auxiliaries and additives such as, for example, antibactericides, isotonicity agents, buffer substances, and/or surfactants=“solvent” below) in a two-component system. A two-chamber system, for example, is described in WO2007/038773 A1.

This increases the tolerable temperature range (in the direction both of lower and of higher temperatures). Moreover, there is an increase in the stability toward mechanical stress such as strong shaking, for example. Consequently, longer shelf lives, lower costs for storage and transport, and safer drug use are anticipated.

The simplified dissolution in one step ensures the capacity for the drug to be used by the patient.

### Example 4

### Formulation of Amidated Insulin Derivatives

Examples 4 to 8 serve only for the determination of the biological, pharmacological, and physicochemical properties of insulin analogs of formula I, involving first the provision of formulations thereof (example 4) and then the conduct of corresponding tests (examples 5 to 8). A solution with the compounds was prepared as follows: the insulin analog of the invention was dissolved with a target concentration of 240±5 μM in 1 mM hydrochloric acid with 80 μg/ml zinc (as zinc chloride).

The compositions used as dissolution medium were as follows:
a) 1 mM hydrochloric acid
b) 1 mM hydrochloric acid, 5 μg/ml zinc (added as zinc chloride or hydrochloric acid)
c) 1 mM hydrochloric acid, 10 μg/ml zinc (added as zinc chloride or hydrochloric acid)
d) 1 mM hydrochloric acid, 15 μg/ml zinc (added as zinc chloride or hydrochloric acid)
e) 1 mM hydrochloric acid, 30 μg/ml zinc (added as zinc chloride or hydrochloric acid)
f) 1 mM hydrochloric acid, 80 μg/ml zinc (added as zinc chloride or hydrochloric acid)
g) 1 mM hydrochloric acid, 120 μg/ml zinc (added as zinc chloride or hydrochloric acid)

For this purpose, an amount of the freeze-dried material higher by around 30% than the amount needed on the basis of the molecular weight and the target concentration was first weighed out. Thereafter the existing concentration was determined by means of analytical HPLC and the solution was then made up with 5 mM hydrochloric acid with 80 μg/ml zinc to the volume needed in order to achieve the target concentration. If necessary, the pH was readjusted to 3.5±0.1. Following final analysis by HPLC to ensure the target concentration of 240±5 μM, the completed solution was transferred, using a syringe having a 0.2 μm filter attachment, into a sterile vial which was closed with a septum and a crimped cap. For the short-term, single testing of the insulin derivatives of the invention, there was no optimization of the formulations, in relation, for example, to addition of isotonic agents, preservatives or buffer substances.

### Example 5

### Evaluation of the Blood Sugar-Reducing Action of New Insulin Analogs in Rats

The blood sugar-lowering effect of selected new insulin analogs is tested in healthy male normoglycemic Wistar rats. Male rats receive a subcutaneous injection of a dose of 9 nmol/kg of an insulin analog. Immediately before the injection of the insulin analog and at regular intervals for up to eight hours after injection, blood samples are taken from the animals, and their blood sugar content determined. The experiment shows clearly (cf. FIG. 1) that the insulin analog of the invention that is used leads to a significantly retarded onset of action and to a longer, uniform duration of action.

### Example 6

### Evaluation of the Blood Sugar-Reducing Action of New Insulin Analogs in Dogs

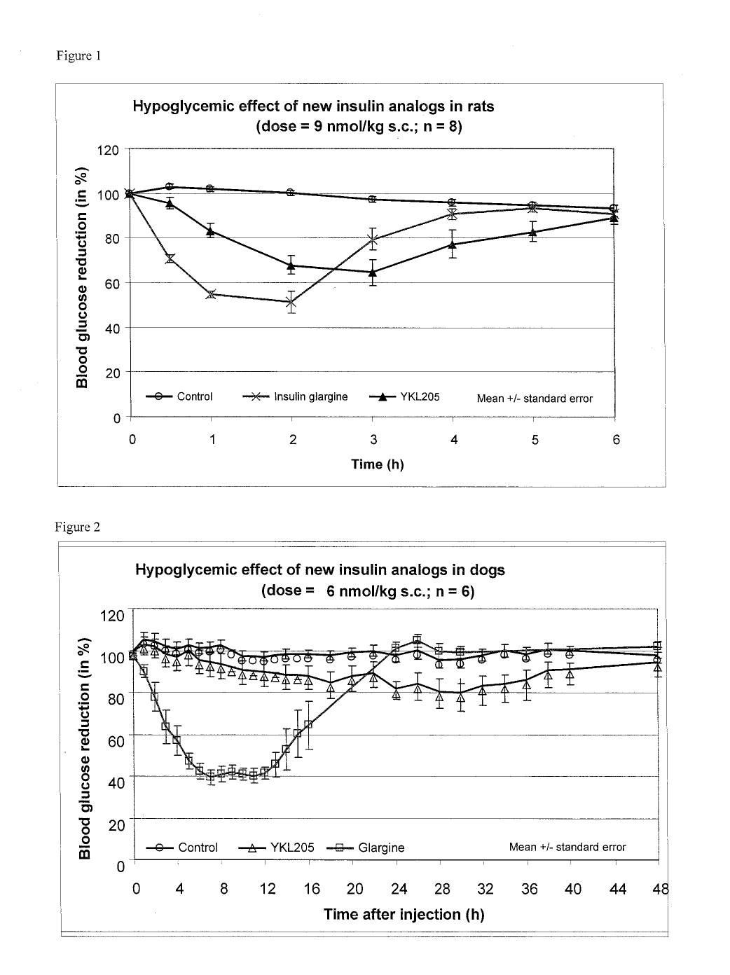

The blood sugar-lowering effect of selected new insulin analogs is tested in healthy male normoglycemic beagles. Male animals receive a subcutaneous injection of a dose of 6 nmol/kg of an insulin analog. Immediately before the injection of the insulin analog and at regular intervals for up to forty-eight hours after injection, blood samples are taken from the animals, and their blood sugar content determined. The experiment shows clearly (cf. FIG. 2) that the insulin analog of the invention that is used leads to a significantly retarded onset of action and to a longer, uniform duration of action.

### Example 7

### Evaluation of the Blood Sugar-Reducing Action in Dogs with Twofold-Increased Dose

The blood sugar-lowering effect of selected new insulin analogs is tested in healthy male normoglycemic beagles. Male animals receive a subcutaneous injection of a dose of 6 nmol/kg and 12 nmol/kg of an insulin analog. Immediately before the injection of the insulin analog and at regular intervals for up to forty-eight hours after injection, blood samples are taken from the animals, and their blood sugar content determined.

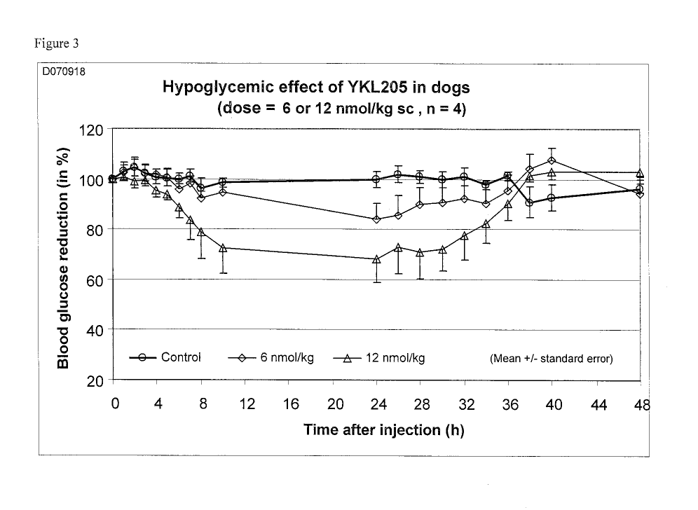

The experiment shows clearly (cf. FIG. 3) that the insulin analog of the invention that is used has a dose-dependent effect, but that, despite the twofold-increased dose, the effect profile is flat, i.e., there is no pronounced low point (nadir) observed. From this it may be inferred that the insulins of the invention, in comparison to known retarded insulins, lead to significantly fewer hypoglycemic events.

### Example 8

### Evaluation of the Blood Sugar-Reducing Effect in Dogs with Different Concentrations of Zinc in the Formulation

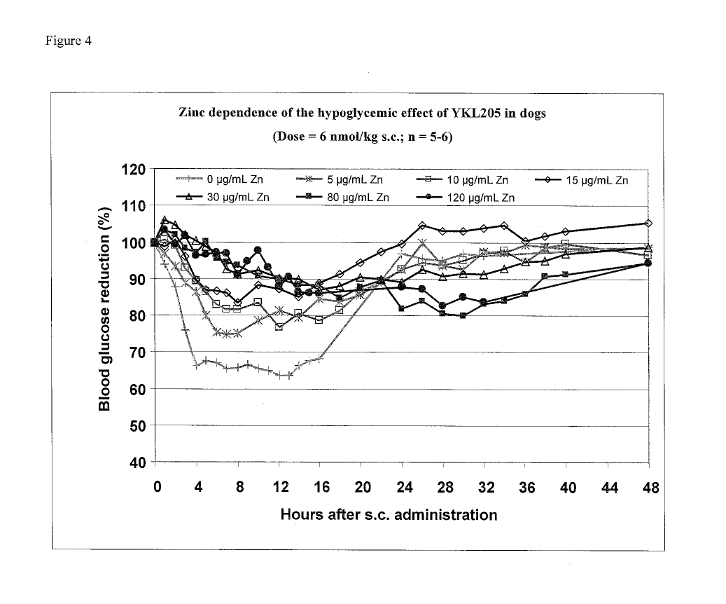

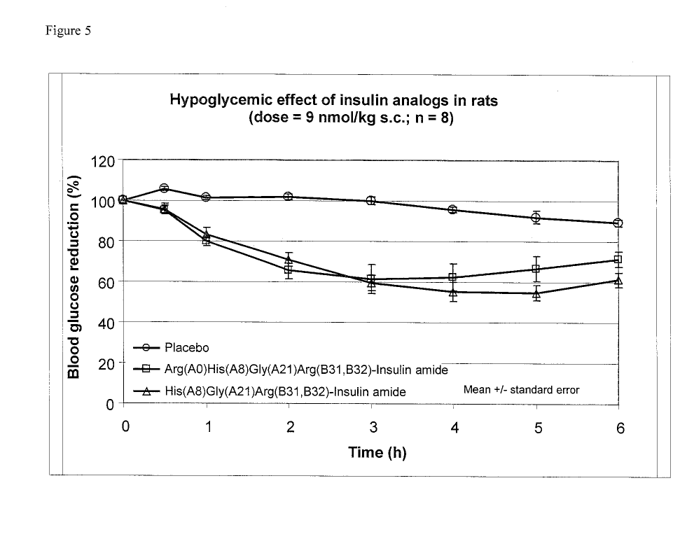

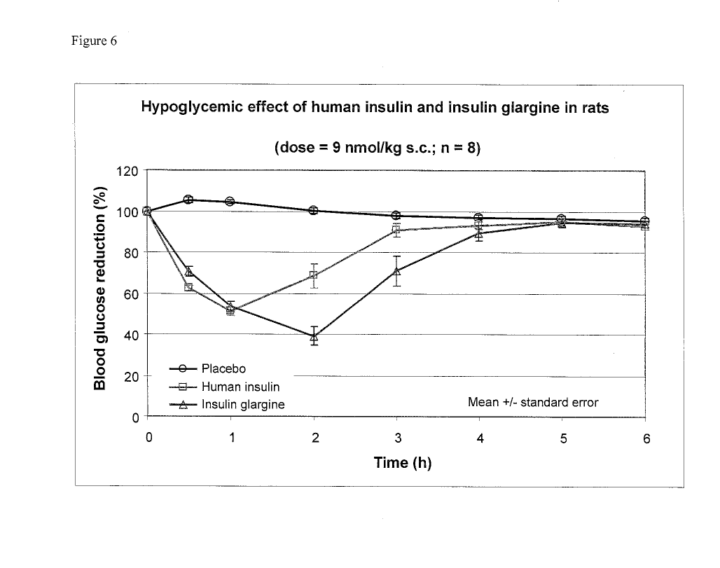

The experiments were carried out as described in example 35. FIG. 4 shows the result. Accordingly, the time/activity curve of the insulin analog of the invention can be influenced through the amount of zinc ions in the formulation, with the same concentration of insulin, in such a way that a rapid onset of action is observed at zero or low zinc content and the action persists over 24 hours, whereas, with a higher zinc content, a flat onset of action is observed and the insulin effect persists for much longer than 24 hours.

### Example 9

### Formulation of Amidated Insulin Derivatives

Examples 9 to 11 serve only for the determination of the biological, pharmacological, and physicochemical properties of insulin analogs of formula II, involving first the provision of formulations thereof (example 9) and then the conduct of corresponding tests (examples 10 and 11). The insulin analog of the invention was dissolved with a target concentration of 240±5 μM in 1 mM hydrochloric acid with 80 μg/ml zinc (as zinc chloride). For this purpose, an amount of the freeze-dried material higher by around 30% than the amount needed on the basis of the molecular weight and the target concentration was first weighed out. Thereafter the existing concentration was determined by means of analytical HPLC and the solution was then made up with 5 mM hydrochloric acid with 80 μg/ml zinc to the volume needed in order to achieve the target concentration. If necessary, the pH was readjusted to 3.5±0.1. Following final analysis by HPLC to ensure the target concentration of 240±5 μM, the completed solution was transferred, using a syringe having a 0.2 μm filter attachment, into a sterile vial which was closed with a septum and a crimped cap. For the short-term, single testing of the insulin derivatives of the invention, there was no optimization of the formulations, in relation, for example, to addition of isotonic agents, preservatives or buffer substances.

### Example 10

### Evaluation of the Blood Sugar-Reducing Action of New Insulin Analogs in Rats

The blood sugar-lowering effect of selected new insulin analogs is tested in healthy male normoglycemic Wistar rats. Male rats receive a subcutaneous injection of a dose of 9 nmol/kg of an insulin analog. Immediately before the injection of the insulin analog and at regular intervals for up to eight hours after injection, blood samples are taken from the animals, and their blood sugar content determined. The experiment shows clearly (cf. FIG. 5) that the insulin analog of the invention leads to a significantly retarded onset of action and to a longer, uniform duration of action.

### Example 11

### Evaluation of the Blood Sugar-Reducing Action of New Insulin Analogs in Dogs

The blood sugar-lowering effect of selected new insulin analogs is tested in healthy male normoglycemic beagles. Male animals receive a subcutaneous injection of a dose of 6 nmol/kg of an insulin analog. Immediately before the injection of the insulin analog and at regular intervals for up to forty-eight hours after injection, blood samples are taken from the animals, and their blood sugar content determined. The experiment shows clearly that the insulin analog of the invention leads to a significantly retarded, flat onset of action and to a longer, uniform duration of action.

1. A method for preparing an aqueous pharmaceutical formulation comprising an insulin, insulin analog or insulin derivative, or a pharmacologically tolerable salt thereof, the method comprising dissolving said insulin, insulin analog or insulin derivative in solid form with a suitable solvent mixture.

2. The method as claimed in claim 1, wherein said suitable solvent mixture is determined by
(a) preparing solvent mixtures, differing in pH and having excipient concentrations corresponding to a final excipient concentration of said formulation that comprises the insulin, insulin analog or insulin derivative,
(b) dissolving the insulin, insulin analog or insulin derivative in each solvent mixture, and
(c) determining which solvent mixture, following dissolution of the solid of insulin, insulin analog or insulin derivative, produces the desired pH of the formulation.

3. The method as claimed in claim 1, wherein the insulin, insulin analog or insulin derivative takes the form of a crystalline or amorphous solid.

4. The method as claimed in claim 1, wherein the insulin is selected from the group consisting of human insulin, porcine insulin and bovine insulin.

5. The method as claimed in claim 1, wherein the insulin analog is selected from the group consisting of Gly(A21), Arg(B31), Arg(B32) human insulin, Lys(B3), Glu(B29) human insulin, Asp(B28) human insulin, Lys(B28) Pro(B29) human insulin and Des(B30) human insulin.

6. The method as claimed in claim 1, wherein the insulin analog is selected from the group consisting of an insulin analog of the formula I 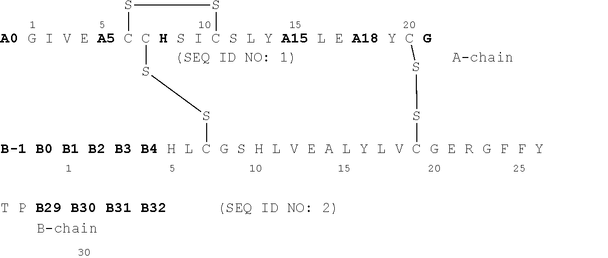
where
A0 is Lys or Arg;
A5 is Asp, Gln or Glu;
A15 is Asp, Glu or Gln;
A18 is Asp, Glu or Asn;
B-1 is Asp, Glu or an amino group;
B0 is Asp, Glu or a chemical bond;
B1 is Asp, Glu or Phe;
B2 is Asp, Glu or Val;
B3 is Asp, Glu or Asn;
B4 is Asp, Glu or Gln;
B29 is Lys or a chemical bond;
B30 is Thr or a chemical bond;
B31 is Arg, Lys or a chemical bond;
B32 is Arg-amide, Lys-amide or an amino group,
where two amino acid residues of the group containing A5, A15, A18, B-1, B0, B1, B2, B3, and B4, simultaneously and independently of one another, are Asp or Glu, and pharmacologically tolerable salts thereof.

7. The method as claimed in claim 6, wherein the insulin analog is selected from the group consisting of:
Arg (A0), His (A8), Glu (A5), Asp (A18), Gly (A21), Arg (B31), Arg (B32)-NH₂ human insulin,
Arg (A0), His (A8), Glu (A5), Asp (A18), Gly (A21), Arg (B31), Lys (B32)-NH₂ human insulin,
Arg (A0), His (A8), Glu (A15), Asp (A18), Gly (A21), Arg (B31), Arg (B32)-NH₂ human insulin,
Arg (A0), His (A8), Glu (A15), Asp (A18), Gly (A21), Arg (B31), Lys (B32)-NH₂ human
Arg (A0), His (A8), Glu(A5), Glu (A15), Gly (A21), Arg (B31), Arg (B32)-NH₂ human insulin,
Arg (A0), His (A8), Glu (A5), Glu (A15), Gly (A21), Arg (B31), Lys (B32)-NH₂ human insulin,
Arg (A0), His (A8), Glu (A5), Gly (A21), Asp (B3), Arg (B31), Arg (B32)-NH₂ human insulin,
Arg (A0), His (A8), Glu (A5), Gly (A21), Asp (B3), Arg (B31), Lys (B32)-NH₂ human insulin,
Arg (A0), His (A8), Glu (A15), Gly (A21), Asp (B3), Arg (B31), Arg (B32)-NH₂ human insulin,
Arg (A0), His (A8), Glu (A15), Gly (A21), Asp (B3), Arg (B31), Lys (B32)-NH₂ human insulin,
Arg (A0), His (A8), Asp (A18), Gly (A21), Asp (B3), Arg (B31), Arg (B32)-NH₂ human insulin,
Arg (A0), His (A8), Asp (A18), Gly (A21), Asp (B3), Arg (B31), Lys (B32)-NH₂ human insulin,
Arg (A0), His (A8), Gly (A21), Asp (B3), Glu (B4), Arg (B31), Arg (B32)-NH₂ human insulin,
Arg (A0), His (A8), Gly (A21), Asp (B3), Glu (B4), Arg (B31), Lys (B32)-NH₂ human insulin,
Arg (A0), His (A8), Glu (A5), Gly (A21), Glu (B4), Arg (B31), Arg (B32)-NH₂ human insulin,
Arg (A0), His (A8), Glu (A5), Gly (A21), Glu (B4), Arg (B31), Lys (B32)-NH₂ human insulin,
Arg (A0), His (A8), Glu (A15), Gly (A21), Glu (B4), Arg (B31), Arg (B32)-NH₂ human insulin,
Arg (A0), His (A8), Glu (A15), Gly (A21), Glu (B4), Arg (B31), Lys (B32)-NH₂ human insulin,
Arg (A0), His (A8), Asp (A18), Gly (A21), Glu (B4), Arg (B31), Arg (B32)-NH₂ human insulin,
Arg (A0), His (A8), Asp (A18), Gly (A21), Glu (B4), Arg (B31), Lys (B32)-NH₂ human insulin,
Arg (A0), His (A8), Glu (A5), Gly (A21), Glu (B0), Arg (B31), Arg (B32)-NH₂ human insulin,
Arg (A0), His (A8), Glu (A5), Gly (A21), Glu (B0), Arg (B31), Lys (B32)-NH₂ human insulin,
Arg (A0), His (A8), Glu (A15), Gly (A21), Glu (B0), Arg (B31), Arg (B32)-NH₂ human insulin,
Arg (A0), His (A8), Glu (A15), Gly (A21), Glu (B0), Arg (B31), Lys (B32)-NH₂ human insulin,
Arg (A0), His (A8), Asp (A18), Gly (A21), Glu (B0), Arg (B31), Arg (B32)-NH₂ human insulin,
Arg (A0), His (A8), Asp (A18), Gly (A21), Glu (B0), Arg (B31), Lys (B32)-NH₂ human insulin,
Arg (A0), His (A8), Glu (A5), Gly (A21), Asp (B1), Arg (B31), Arg (B32)-NH₂ human insulin,
Arg (A0), His (A8), Glu (A5), Gly (A21), Asp (B1), Arg (B31), Lys (B32)-NH₂ human insulin,
Arg (A0), His (A8), Glu (A15), Gly (A21), Asp (B1), Arg (B31), Arg(B32)-NH₂ human insulin,
Arg (A0), His (A8), Glu (A15), Gly (A21), Asp (B1), Arg (B31), Lys (B32)-NH₂ human insulin,
Arg (A0), His (A8), Asp (A18), Gly (A21), Asp (B1), Arg (B31), Arg (B32)-NH₂ human insulin,
Arg (A0), His (A8), Asp (A18), Gly (A21), Asp (B1), Arg (B31), Lys (B32)-NH₂ human insulin,
Arg (A0), His (A8), Gly (A21), Glu (B0), Asp (B1), Arg (B31), Arg (B32)-NH₂ human insulin,
Arg (A0), His (A8), Gly (A21), Glu (B0), Asp (B1), Arg (B31), Lys (B32)-NH₂ human insulin,
Arg (A0), His (A8), Asp (A18), Gly (A21), Asp (B3), Arg (B30), Arg (B31)-NH₂ human insulin and
Arg (A0), His (A8), Asp (A18), Gly (A21), Asp (B3), Arg (B30), Lys (B31)-NH₂ human insulin.

8. The method as claimed in claim 1, wherein the insulin analog is
an insulin analog of the formula II 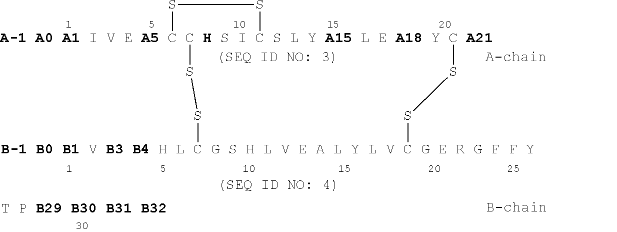
where
A-1 is Lys, Arg or an amino group;
A0 is Lys, Arg or a chemical bond;
A1 is Arg or Gly;
A5 is Asp, Glu or Gln;
A15 is Asp, Glu or Gln;
A18 is Asp, Glu or Asn;
A21 is Ala, Ser, Thr or Gly;
B-1 is Asp, Glu or an amino group;
B0 is Asp, Glu or a chemical bond;
B1 is Asp, Glu, Phe or a chemical bond;
B3 is Asp, Glu or Asn;
B4 is Asp, Glu or Gln;
B29 is Arg, Lys or an amino acid selected from the group containing the amino acids Phe, Ala, Thr, Ser, Val, Leu, Glu or Asp, or a chemical bond;
B30 is Thr or a chemical bond;
B31 is Arg, Lys or a chemical bond;
B32 is Arg-amide or Lys-amide,
where not more than one amino acid residue from the group containing A5, A15, A18, B-1, B0, B1, B2, B3 and B4, simultaneously and independently of one another, is Asp or Glu.

9. The method as claimed in claim 6, wherein the insulin analog is selected from the group consisting of:
Arg (A-1), Arg (A0), Glu (A5), His (A8), Gly (A21), Arg (B30)-NH₂ human insulin,
Arg (A-1), Arg (A0), Glu (A5), His (A8), Gly (A21), Lys (B30)-NH₂ human insulin,
Arg (A-1), Arg (A0), Glu (A15), His (A8), Gly (A21), Arg (B30)-NH₂ human insulin,
Arg (A-1), Arg (A0), Glu (A15), His (A8), Gly (A21), Lys (B30)-NH₂ human insulin,
Arg (A-1), Arg (A0), Asp (A18), His (A8), Gly (A21), Arg (B30)-NH₂ human insulin,
Arg (A-1), Arg (A0), Asp (A18), His (A8), Gly (A21), Arg (B30)-NH₂ human insulin,
Arg (A-1), Arg (A0), His (A8), Gly (A21), Glu (B0), Arg (B30)-NH₂ human insulin,
Arg (A-1), Arg (A0), His (A8), Gly (A21), Glu (B0), Lys (B30)-NH₂ human insulin,
Arg (A-1), Arg (A0), His (A8), Gly (A21), Asp (B3), Arg (B30)-NH₂ human insulin,
Arg (A-1), Arg (A0), His (A8), Gly (A21), Asp (B3), Lys (B30)-NH₂ human insulin,
Arg (A-1), Arg (A0), His (A8), Gly (A21), Glu (B4), Arg (B30)-NH₂ human insulin,
Arg (A-1), Arg (A0), His (A8), Gly (A21), Glu (B4), Lys (B30)-NH₂ human insulin,
Arg (A0), His (A8), Gly (A21), Arg (B31), Arg (B32)-NH₂ human insulin,
Arg (A0), His (A8), Gly (A21), Arg (B31), Lys (B32)-NH₂ human insulin,
Arg (A0), Glu (A5), His (A8), Gly (A21), Arg (B31), Arg (B32)-NH₂ human insulin,
Arg (A0), Glu (A5), His (A8), Gly (A21), Arg (B31), Lys (B32)-NH₂ human insulin,
Arg (A0), Asp (A18), His (A8), Gly (A21), Arg (B31), Arg (B32)-NH₂ human insulin,
Arg (A0), Asp (A18), His (A8), Gly (A21), Arg (B31), Lys (B32)-NH₂ human insulin,
Arg (A0), Glu (A15), His (A8), Gly (A21), Arg (B31), Arg (B32)-NH₂ human insulin,
Arg (A0), Glu (A15), His (A8), Gly (A21), Arg (B31), Lys (B32)-NH₂ human insulin,
Arg (A0), His (A8), Gly (A21), Asp (B3), Arg (B31), Arg (B32)-NH₂ human insulin,
Arg (A0), His (A8), Gly (A21), Asp (B3), Arg (B31), Lys (B32)-NH₂ human insulin,
Arg (A0), His (A8), Gly (A21), Glu (B4), Arg (B31), Arg (B32)-NH₂ human insulin,
Arg (A0), His (A8), Gly (A21), Glu (B4), Arg (B31), Lys (B32)-NH₂ human insulin,
Arg (A0), His (A8), Gly (A21), Glu (B0), Arg (B31), Arg (B32)-NH₂ human insulin,
Arg (A0), His (A8), Gly (A21), Glu (B0), Arg (B31), Lys (B32)-NH₂ human insulin,
Arg (A0), His (A8), Gly (A21), Arg (B30)-NH₂ human insulin,
Arg (A0), His (A8), Gly (A21), Lys (B30)-NH₂ human insulin,
Arg (A-1), Arg (A0), His (A8), Gly (A21), Arg (B30)-NH₂ human insulin,
Arg (A-1), Arg (A0), His (A8), Gly (A21), Lys (B30)-NH₂ human insulin,
Arg (A0), Arg (A1), His (A8), Gly (A21), Arg (B30)-NH₂ human insulin,
Arg (A0), Arg (A1), His (A8), Gly (A21), Lys (B30)-NH₂ human insulin and
His (A8), Gly (A21), Arg (B31), Arg (B32)-NH₂ human insulin.

10. The method as claimed in claim 1, wherein the insulin derivative is selected from the group consisting of B29-N-myristoyl-des(B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28} Pro^{B29} human insulin, B30-N-myristoyl-Thr^{B29}Lys^{B30} human insulin, B30-N-palmitoyl-Thr^{B29}Lys^{B30} human insulin, B29-N-(N-palmitoyl-Y-glutamyl)-des(B39) human insulin, B29-N-(N-lithocholyl-Y-glutamyl)-des(B30) human insulin, B29-N-(ω-carboxyheptadecanoyl)-des(B30) human insulin and B29-N-(ω-carboxyheptadecanoyl) human insulin.

11. The method as claimed in claim 1, wherein said formulation comprises a preservative selected from the group consisting of phenol, m-cresol, chlorocresol, benzyl alcohol and parabens.

12. The method as claimed in claim 1, wherein said formulation comprises an isotonicity agent selected from the group consisting of mannitol, sorbitol, lactose, dextrose, trehalose, sodium chloride and glycerol.

13. The method as claimed in claim 1, wherein said insulin, insulin analog and/or insulin derivative is present in a concentration of 240-3000 nmol/ml.

14. The method as claimed in claim 1, wherein said formulation further comprises a glucagon-like peptide-1 (GLP1) or an analog or derivative thereof, or exendin-3 and/or -4 or an analog or derivative thereof.

15. The method as claimed in claim 14, wherein said analog of exendin-4 is selected from the group consisting of
H-desPro³⁶-exendin-4-Lys₆-NH₂,
H-des(Pro^{36,37})-exendin-4-Lys₄-NH₂,
H-des(Pro^{36,37})-exendin-4-Lys₅-NH₂, and
pharmacologically tolerable salts thereof.

16. The method as claimed in claim 14, wherein said analog of exendin-4 is selected from the group consisting of
desPro³⁶ [Asp²⁸]exendin-4 (1-39),
desPro³⁶ [IsoAsp²⁸]exendin-4 (1-39),
desPro³⁶ [Met(O)¹⁴, Asp²⁸]exendin-4 (1-39),
desPro³⁶ [Met(O)¹⁴, IsoAsp²⁸]exendin-4 (1-39),
desPro³⁶ [Trp(O₂)²⁵, Asp²⁸]exendin-2 (1-39),
desPro³⁶ [Trp(O₂)²⁵, IsoAsp²⁸]exendin-2 (1-39),
desPro³⁶ [Met(O)¹⁴Trp(O₂)²⁵, Asp²⁸]exendin-4 (1-39) and
desPro³⁶ [Met(O)¹⁴Trp(O₂)²⁵, IsoAsp²⁸]exendin-4 (1-39), and
pharmacologically tolerable salts thereof.

17. The method as claimed in claim 16, wherein said peptide Lys₆-NH₂ is attached to the C-termini of the analogs of exendin-4.

18. The method as claimed in claim 14, wherein analog of exendin-4 is selected from the group consisting of
H-(Lys)₆-des Pro³⁶ [Asp²⁸]exendin-4(1-39)-Lys₆-NH₂
des Asp²⁸Pro³⁶, Pro³⁷, Pro³⁸ exendin-4(1-39)-NH₂,
H-(Lys)₆-des Pro³⁶, Pro³⁷, Pro³⁸ [Asp²⁸]exendin-4(1-39)-NH₂,
H-Asn-(Glu)₅ des Pro³⁶, Pro³⁷, Pro³⁸ [Asp²⁸]exendin-4(1-39)-NH₂,
des Pro³⁶, Pro³⁷, Pro³⁸ [Asp²⁸]exendin-4(1-39)-(Lys)₆-NH₂,
H-(Lys)₆-des Pro³⁶, Pro³⁷, Pro³⁸ [Asp²⁸]exendin-4(1-39)-(Lys)₆-NH₂,
H-Asn-(Glu)₅-des Pro³⁶, Pro³⁷, Pro³⁸ [Asp²⁸]exendin-4(1-39)-(Lys)₆-NH₂,
H-(Lys)₆-des Pro³⁶ [Trp(O₂)²⁵, Asp²⁸]exendin-4(1-39)-Lys₆-NH₂,
H-des Asp²⁸ Pro³⁶, Pro³⁷, Pro³⁸ [Trp(O₂)²⁵]exendin-4(1-39)-NH₂,
H-(Lys)₆-des Pro³⁶, Pro³⁷, Pro³⁸ [Trp(O₂)²⁵, Asp²⁸]exendin-4(1-39)-NH₂,
H-Asn-(Glu)₅-des Pro³⁶, Pro³⁷, Pro³⁸ [Trp(O₂)²⁵, Asp²⁸]exendin-4(1-39)-NH₂,
des Pro³⁶, Pro³⁷, Pro³⁸ [Trp(O₂)²⁵, Asp²⁸]exendin-4(1-39)-(Lys)₆-NH₂,
H-(Lys)₆-des Pro³⁶, Pro³⁷, Pro³⁸ [Trp(O₂)²⁵, Asp²⁸]exendin-4(1-39)-(Lys)₆-NH₂,
H-Asn-(Glu)₅-des Pro³⁶, Pro³⁷, Pro³⁸ [Trp(O₂)²⁵, Asp²⁸]exendin-4(1-39)-(Lys)₆-NH₂,
H-(Lys)₆-des Pro³⁶ [Met(O)¹⁴, Asp²⁸]exendin-4(1-39)-Lys₆-NH₂,
des Met(O)¹⁴ Asp²⁸ Pro³⁶, Pro³⁷, Pro³⁸ exendin-4(1-39)-NH₂,
H-(Lys)₆-des Pro³⁶, Pro³⁷, Pro³⁸ [Met(O)¹⁴, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Pro³⁷, Pro³⁸ [Met(O)¹⁴, Asp²⁸]exendin-4(1-39)-(Lys)₆-NH₂,
H-(Lys)₆-des Pro³⁶, Pro³⁷, Pro³⁸ [Met(O)¹⁴, Asp²⁸]exendin-4(1-39)-Lys₆-NH₂,
H-Asn-(Glu)₅ des Pro³⁶, Pro³⁷, Pro³⁸ [Met(O)¹⁴, Asp²⁸]exendin-4(1-39)-(Lys)₆-NH₂,
H-(Lys)₆-des Pro³⁶ [Met(O)¹⁴, Trp(O₂)²⁵, Asp²⁸]exendin-4(1-39)-Lys₆-NH₂,
des Asp²⁸ Pro³⁶, Pro³⁷, Pro³⁸ [Met(O)¹⁴, Trp(O₂)²⁵]exendin-4(1-39)-NH₂,
H-(Lys)₆-des Pro³⁶, Pro³⁷, Pro³⁸ [Met(O)¹⁴, Trp(O₂)²⁵, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Pro³⁷, Pro³⁸ [Met(O)¹⁴, Trp(O₂)²⁵, Asp²⁸]exendin-4(1-39)-(Lys)₆-NH₂,
H-(Lys)₆-des Pro³⁶, Pro³⁷, Pro³⁸ [Met(O)¹⁴, Trp(O₂)²⁵, Asp²⁸]exendin-4(1-39)-(Lys)₆-NH₂,
H-Asn-(Glu)₅-des Pro³⁶, Pro³⁷, Pro³⁸ [Met(O)¹⁴, Trp(O₂)²⁵, Asp²⁸]exendin-4(1-39)-(Lys)₆-NH₂, and
pharmacologically tolerable salts thereof.

19. The method as claimed in claim 14, the formulation further comprising Arg³⁴, Lys²⁶ (N^{ε}(γ-glutamyl(N^{α}-hexadecanoyl))) GLP-1 (7-37) [liraglutide] or a pharmacologically tolerable salt thereof.

20. The method as claimed in claim 1, wherein said formulation further comprises a zinc salt.

21. (canceled)

22. A two-part set of containers, wherein one of the containers contains an insulin, insulin analog or insulin derivative in solid form and the other container contains a solvent mixture having a defined pH and having a final excipient concentration of a desired formulation of insulin, insulin analog or insulin derivative, for the heat-stable and vibration-stable preservation of the insulin, insulin analog or insulin derivative for the later preparation of a ready-to-use formulation by dissolving of the solid in the solvent mixture, as claimed in claim 1.

23. A two-chamber injection system, in which one chamber contains an insulin, insulin analog or insulin derivative in solid form and the other chamber contains a solvent mixture having a defined pH and having a final excipient concentration of a desired formulation of an insulin, insulin analog or insulin derivative, for the heat-stable and vibration-stable preservation of the insulin, insulin analog or insulin derivative for the later preparation of a ready-to-use formulation by dissolving of the solid in the solvent mixture, as claimed in claim 1.
